(19) United States
(12) Patent Application Publication
(10) Pub. No.: US 2010/0233144 A1
(43) Pub. Date: Sep. 16, 2010

(54) METHOD FOR OPTIMIZING BLOOD CELL TRANSPLANTS

(75) Inventors: David Klatzmann, Paris (FR); Jose Cohen, Paris (FR)

Correspondence Address: OCCHIUTI ROHLICEK & TSAO, LLP, 10 FAWCETT STREET, CAMBRIDGE, MA, 02138, US

(73) Assignees: UNIVERSITE PIERRE ET MARIE CURIE (PARIS 6), PARIS (FR); ASSITANCE PUBLIQUE - HOSPITAUX DE PARIS, PARIS (FR); CENTRE NATIONAL DE LA RECHERCHE SCIENTIFIQUE, PARIS (FR)

(21) Appl. No.: 12/682,643

(22) PCT Filed: Oct. 10, 2008
(86) PCT No.: PCT/FR08/51844

§ 371 Date: Apr. 12, 2010

Related U.S. Application Data
(60) Provisional application No. 60/960,761, filed on Oct. 12, 2007.

(51) Int. Cl. A61K 35/26; A61P 7/00

(52) U.S. Cl. 424/93.21; 424/93.71

(57) ABSTRACT

The invention relates to the use of allogenic T lymphocytes for the preparation of a composition intended to be injected into a recipient patient as a conditioning for a transplantation of haematopoietic stem cells, said allogenic T lymphocytes expressing a molecule allowing their specific destruction.

The present invention relates to the field of transplantations of haematopoietic stem cells. More precisely, this invention relates to the conditioning of recipients of haematopoietic stem cells with a composition containing T lymphocytes in order to optimize the transplantation of haematopoietic stem cells.

Haematopoietic stem cells are cells which are responsible for all the blood cell lines. These cells are capable of giving rise, through differentiation, to any blood cell (red blood cells, white blood cells, platelets) and are also capable of self-renewal.

Transplantation of haematopoietic stem cells has become over the past few years one of the major therapeutic means in the treatment of some blood diseases and of some cancers. Indeed, it allows a therapeutic intensification through chemotherapy and/or radiotherapy at massive doses resulting in the treatment of the disease, or even its cure with an improvement in the survival of the patient.

A transplantation performed after these treatments which have a high haematological toxicity allows the reconstruction of the bone marrow and the return to a normal production of blood cells.

The transplantation of haematopoietic stem cells occurs in the treatment of several types of pathology: malignant such as acute leukaemias, myelomas, lymphomas or certain solid tumours (breast cancer, neuroblastoma) but also non-malignant such as constitutive or acquired deficiencies (immune deficiency, aplasias, metabolic errors). There are three sources of haematopoietic stem cells: bone marrow, peripheral blood and placental blood. Two types of transplantation may be performed:
autotransplantation where the patient receives their own stem cells collected from the bone marrow, the cord blood or from the peripheral blood several weeks, months or years beforehand and stored frozen.
allotransplantation which requires a familial or non-related donor.

The transplantation is performed in several steps. A few days before undergoing the transplantation, the patient is generally subjected to a myeloablative or non-myeloablative “conditioning”. This is often a strong chemotherapy and optionally a full irradiation in order to destroy the marrow of the recipient and allow establishment of the graft. The aim is to destroy all the cells present in the medullary cavities of the recipient in order to allow the haematopoietic stem cells of the donor to develop therein and thus replace the destroyed bone marrow of the recipient.

The transplantation of haematopoietic cells is then performed by intravenous transfusion.

The transplantation is followed by a period of aplasia during which the patient no longer has immune defences.

The “conditioning” period preceding the transplantation makes it possible to obtain an antitumour effect on malignant cells and to create a sufficient immunosuppression in order to prevent the rejection of the graft. An important effect in the establishment of a graft is linked to the presence or absence of allogenic T lymphocytes in the graft. These T lymphocytes favour grafting, via an allogenic activation promoting the establishment of the graft by destroying residual immunocompetent cells of the recipient, thus avoiding their action of rejection of the graft cells. However, this beneficial effect is counterbalanced by the high risks of graft versus host (GVH) disease. It is the main and limiting complication of the transplantation of allogenic marrow. Although the mechanism is still partially not understood, GVH is linked to the activation of the mature T lymphocytes which recognize the host antigens as foreign antigens. The cytotoxic activity which results therefrom is then direct (action of the cytotoxic lymphocytes) or indirect through the recruitment of other effector cells or secretion of cytokines. Acute GVH generally occurs during the 2 to 5 weeks which follow the transplantation and may have a frequency of between 10 and 80% of the cases according to the genetic disparity between donor and recipient. Reference is made to chronic GVH when it occurs more than 100 days after the transplantation. A solution normally used to prevent GVH is to administer to the patient the immunosuppressive molecule cyclosporin A (CsA) 3 to 6 months following the transplantation of haematopoietic stem cells (Storb R et al, 1989). However, the treatment is only partially effective because 15 to 60% of the patients will develop GVH (Socie et al, 1998). The use of other immunosuppressive molecules such as FK506 did not induce the reduction in the frequency of GVH (Ratanatharathorn et al, 1998). Another strategy for preventing GVH is to perform a transplantation of haematopoietic stem cells depleted of T lymphocytes (Aversa et al 1998). The major disadvantage of this strategy is that it does not allow sufficient reconstitution of the T lymphocytes, which generates complications due to infections. Another solution has been proposed in the context of an allogenic bone marrow graft, consisting in supplementing the graft of T lymphocytes expressing the suicide gene of thymidine kinase (TK) which allows the removal, using gancyclovir, of the T lymphocytes expressing it (Cohen et al 2001). In this case, gancyclovir was administered 7 days after the bone marrow transplantation comprising T lymphocytes transduced with the thymidine kinase gene.

### SUMMARY OF THE INVENTION

The inventors now propose using, during the “conditioning” of the patient before transplantation of haematopoietic stem cells, T lymphocytes expressing a molecule allowing their specific destruction, with the aim of promoting the establishment of the graft.

A subject of the invention is the use of allogenic T lymphocytes for the preparation of a composition intended to be injected into a recipient patient before a (as a conditioning for a) transplantation of haematopoietic stem cells, the said allogenic T lymphocytes expressing a molecule allowing their specific destruction.

Another subject of the invention relates to a composition of allogenic T lymphocytes for injection into a recipient patient before a (as a conditioning for a) transplantation of haematopoietic stem cells, the said allogenic T lymphocytes expressing a molecule allowing their specific destruction.

Also described is a method for preventing the development of GVH disease in a patient who is about to undergo a transplantation of haematopoietic stem cells, the method comprising:
the conditioning of the patient by a myeloablative or non-myeloablative lymphopenic treatment,
combined with an injection of T lymphocytes expressing a molecule allowing their specific destruction;
then, optionally, the destruction of the T lymphocytes within three days.

This method makes it possible to reduce the risk of development of GVH disease. It also makes it possible to optimize the conditioning of the recipient and therefore to promote the establishment of the graft, and to inject T lymphocytes which may be obtained from a genetic pool different from that of the donor and of the recipient.

The method makes it possible moreover to use a smaller quantity of haematopoietic stem cells, making possible the use of stem cells of a single cord blood.

### DETAILED DESCRIPTION OF THE INVENTION

### Definitions

The T lymphocytes express a “molecule allowing their specific destruction”. This may be a molecule encoded by a transgene or a molecule that is naturally expressed by the T lymphocytes. The term “specific destruction” means that only the T lymphocytes administered to the patient will be destroyed, to prevent the development of a GVH reaction.

The “molecule allowing their specific destruction” may be for example an antigen of the HLA system, the molecules Thy-1, NGF receptor or a truncated form of the receptor, or else an antigen that is not immunogenic and not naturally expressed by the T lymphocytes. The T lymphocytes carrying either of these molecules can then be specifically destroyed by an antilymphocyte serum, or antibodies specifically directed against said antigens.

The “molecule allowing the specific destruction” of the T lymphocytes may also be a molecule encoded by a “suicide gene”.

The term “suicide gene” refers to a gene encoding a molecule that is toxic for the cell expressing it, conditionally.

### Sources of T Lymphocytes

The allogenic T lymphocytes may be obtained from the donor of HSC or from a different donor, who is neither the donor nor the recipient.

The donor of T lymphocytes is preferably a human being, and may be a foetus, a newborn, a child or an adult.

The preparations of T lymphocytes are obtained for example from peripheral blood, the blood product of a lymphapheresis, peripheral lymph nodes, the spleen, the thymus, the cord blood, and the like.

Injection of the T lymphocytes is performed in order to prepare the transplantation of haematopoietic stem cells. In a preferred embodiment, the injection of T lymphocytes is performed from 0 to 30 days, preferably from 1 to 15 days, and more preferably from 3 to 10 days before the transplantation of haematopoietic stem cells.

### Transduction of Molecules Allowing the Specific Destruction of the T Lymphocytes:

In a specific embodiment, the T lymphocytes are modified so as to express a transgene encoding a molecule allowing the specific destruction of said T lymphocytes. Preferentially, the transgene is a “suicide” gene. For example, it may be a gene which encodes a molecule capable of phosphorylating a nucleoside analogue to a monophosphate molecule, itself convertible by cellular enzymes to a triphosphate nucleotide that can be incorporated into nucleic acids during extension under the effect of polymerases, the effect being the interruption of chain extension. Said nucleotide analogue may be for example acyclovir or gancyclovir. Said molecule expressed by the “suicide” gene may be in particular a thymidine kinase, chosen for example from the thymidine kinase (TK) of the herpes simplex virus type 1, the thymidine kinase of the equine herpes virus, or a truncated thymidine kinase (see in particular patent EP1046400). The thymidine kinase (TK) of the herpes simplex virus type 1 is advantageously used.

The herpes simplex virus 1 thymidine kinase (HSV1-TK) is capable, when it is present in a sufficient concentration in the cells in question, of phosphorylating nucleotide analogues, such as acyclovir (9-[(2-hydroxyethoxy)methyl]guanine) or gancyclovir (9-[1,3-dihydroxy-2-propoxymethyl]guanine), to monophosphate molecules which are themselves convertible by cellular enzymes to triphosphate nucleotides which can be incorporated into nucleic acids during extension under the effect of the polymerases within the said cells, the effect being the interruption of chain extension and cell death which follows.

In case of GVH, the nucleotide analogue (for example gancyclovir) is then administered to the patient.

Advantageously, the nucleoside analogue (for example gancyclovir) is administered, as a prevention, before the transplantation, for example within 3 days after the injection of the T lymphocytes (preferably 3 to 10 hours after).

Use may be made of any suitable technique for transferring the transgene, in particular by in vitro infection of the corresponding cells with an amphotropic Moloney type virus-like particle. These viral particles are produced by a so-called “packaging” cell line which would have been constructed beforehand. A packaging line is capable of manufacturing all the structural elements constituting a viral particle, but is incapable of introducing into viral particles undergoing maturation the viral RNAs produced by this cell line. Accordingly, these so-called packaging lines continuously manufacture empty viral particles.

The introduction of an appropriate genetic construct, which contains the recombinant DNA as defined above, allows these packaging lines to be introduced into the empty viral particles, thus producing virus-like particles. These virus-like particles are capable of infecting various target cells, which target calls vary according to the packaging line which was used at the outset. For example, if this packaging line is derived from a so-called amphotropic Moloney virus, the viral particles produced perfectly infect human haematopoietic cells.

The conventional techniques for the production of cell lines transformed with a retroviral vector (see for example Danos et al., 1988 and Markowitz et al., 1988), may be transposed to the production of lymphocytes. Likewise, the genetic transfer techniques using such systems (Kasid et al, 1990) may be applied to the transfer, in humans, of the cells as defined above. The transfer may also be carried out according to the method described by Lemoine et al. (Efficient transduction and selection of human T-lymphocytes with bicistronic Thy1/HSV1-TK retroviral vector produced by a human packaging cell line, J Gene Med. 2004 Apr.; 6(4):374-86). According to another embodiment of the invention, the T lymphocytes may be genetically modified using a retroviral vector SFCMM-2 encoding the “suicide” gene for HSV-TK/Neo fusion, according to the transduction method described in Ciceri et al., 2007.

### Patient

The intended patient is a human being, regardless of their age and gender, who will be subjected to a transplantation of haematopoietic stem cells. The patient suffers from any disease which may be treated by a transplantation of HSC. This may include in particular cancers, genetic diseases, diseases which affect the haematopoietic system and/or the immune system. There may be mentioned in particular: solid tumours, malignant haemopathies, including chronic myeloid leukaemia, acute myeloid leukaemia, acute lymphoblastic leukaemia, myeloproliferative syndromes, myelodysplasic syndromes, NHLs (non-Hodgkin malignant lymphomas), Hodgkin, idiopathic severe medullary aplasia, paroxysmal nocturnal haemoglobinuria, severe haemoglobinopathies, severe congenital immune deficiencies (SCID, Kostmann, Wiskott-Aldrich) and Fanconi's anaemia.

### Conditioning of the Patient

The conditioning of the patient, associated with the injection of the T lymphocytes expressing the “suicide” gene before the transplantation of haematopoietic stem cells, may be a myeloablative or non-myeloablative lymphopenic treatment.

A review of this type of treatment is presented in Petersen et al, 2007. An example of a myeloablative lymphopenic treatment may be the following: cyclophosphamide (120 mg/kg) combined with busulfan (16 mg/kg) or with full irradiation of the patient with an absorbed dose of ray of 12 Gy (“total body irradiation”).

In the case of the non-myeloablative lymphopenic treatment, the patient may be fully irradiated with an absorbed dose of radiation of 2 Gy or receive a chemotherapy based on cyclophosphamide and/or fludarabine. For example, Miller et al., 2007, describe a non-myeloablative lymphopenic treatment which comprises an intravenous injection of cyclophosphamide 50 mg/kg once at D−6 and D−5, and injection of fludarabine 25 mg/m² from D−6 to D−2 (D0 being the day of the transplantation of haematopoietic stem cells).

### The Haematopoietic Stem Cells

The haematopoietic stem cells used may be obtained from any source, for example from peripheral blood, bone marrow or umbilical cord blood.

Indeed, the method proposed makes it possible to reduce the number of HSC to be injected so that there is establishment of the graft.

In a preferred embodiment, less than 2 to 3×10⁷ nucleated cells of umbilical cord blood per kg of transplant patient are used. When the stem cells are obtained from marrow or peripheral blood, less than 2×10⁸ nucleated cells per kg (of transplanted patient) or less than 2-3×10⁶ CD34+ cells per kg (of transplanted patient) are used.

### Protocol

Since the T lymphocytes express a molecule allowing their specific destruction, the GVH may be controlled. It is therefore possible to use a large quantity of T lymphocytes, for example more than 10⁵ CD3+ cells per kg (of transplanted patient). According to a particular embodiment, the composition to be injected for conditioning the patient comprises modified T lymphocytes obtained by:

i. lymphapheresis of the donor,
ii. transduction, preferably on the same day, of the gene encoding a molecule allowing the specific destruction of the T lymphocytes (for example a “suicide” gene),
iii. then culturing of the T lymphocytes thus modified for ten to 21 days, until about 10⁸ cells per kg of body mass of the patient are obtained.

Between D−10 and D−3, D0 being the day of the transplantation of haematopoietic stem cells, the modified T lymphocyte composition is administered to the patient by injection after a myeloablative or non-myeloablative lymphopenic treatment.

This treatment may be characterized by an irradiation of between 2 and 12 Gy. Furthermore, this irradiation may be combined with the administration of cyclophosphamide (120 mg/kg) combined with busulfan (16 mg/kg) or the irradiation may be combined with cyclophosphamide, fludarabine and/or endoxan. Miller et al, 2007, describe a non-myeloablative lymphopenic treatment which may also be useful. This treatment comprises an IV injection of cyclophosphamide 50 mg/kg once at D−6 and D−5, and injection of fludarabine 25 mg/m² from D−6 to D−2 (D being the day of the transplantation of the T lymphocytes). Powell et al., 2007, describe another protocol comprising the injection of fludarabine 25 mg/m² for 5 days and endoxan 60 mg/kg/day for 2 days.

Next, the nucleoside analogue is administered to the patient, preferably via one or more daily doses from a few hours up to 3 days after the injection of T lymphocytes.

The examples and figures illustrate the invention without limiting its scope.

### LEGEND TO THE FIGURES

FIG. 1 is a diagram showing the percentage expression of the H2 Kb marker in several groups of mice having received different “conditioning” treatments at D+27 (D0 being the day of the transplantation of haematopoietic stem cells). Irr 7Gy means that the mice were subjected to an irradiation of 7 Gy. GMO means bone marrow transplantation. GCV means gancyclovir.

FIG. 2 is a diagram showing the percentage expression of the H2 Kb marker in several groups of mice having received different “conditioning” treatments at D+57. Irr 7Gy means that the mice were subjected to an irradiation of 7 Gy. GMO means bone marrow transplantation. GVC means gancyclovir.

FIG. 3 describes a study of chimerism as a function of the irradiation dose at 1 month.

FIGS. 4 and 5 show the impact of the T lymphocytes expressing the TK gene in the establishment of the graft.

FIG. 6 illustrates a therapeutic protocol with conditioning of the recipient.

### EXAMPLES

Studies were carried out on mice. The mice (B6xD2)F1H2^{-bxd} were used as recipient mice and the mice C57B1/6CD3e−/− were used as donor mice for haematopoietic stem cells. In this experiment, the haematopoietic stem cells are obtained from the bone marrow. All the recipient mice (B6xD2)F1H2^{-bxd} were exposed to an irradiation of the order of 7 Gy. A quantity of 5×10⁵ haematopoietic stem cells derived from the spinal cord was collected from all the donor mice C57B1/6CD3e−/−. A group of recipient mice (B6xD2)F1H2^{-bxd} was subjected to an irradiation of 7 Gy combined with the injection of a composition comprising ±2×10⁶ T lymphocytes C57B1/6TK. The second group of recipient mice (B6xD2)F1H2^{-bxd} was subjected to an irradiation of 7 Gy during its “conditioning” at D−3, without injection of TK T lymphocytes. All the recipient mice (B6xD2)F1H2^{-bxd} were transplanted with a quantity of 5×10⁵ haematopoietic stem cells derived from the spinal cord of the donor mice 72 h after the irradiation. The group of mice (B6xD2)F1H2^{-bxd} having received during its “conditioning” the composition comprising ±(2×10⁶) T lymphocytes C57B1/6TK was subdivided into two subgroups. One of the subgroups was exposed to gancyclovir (nucleoside analogue) at D−3 while the other subgroup received gancyclovir at D−1.

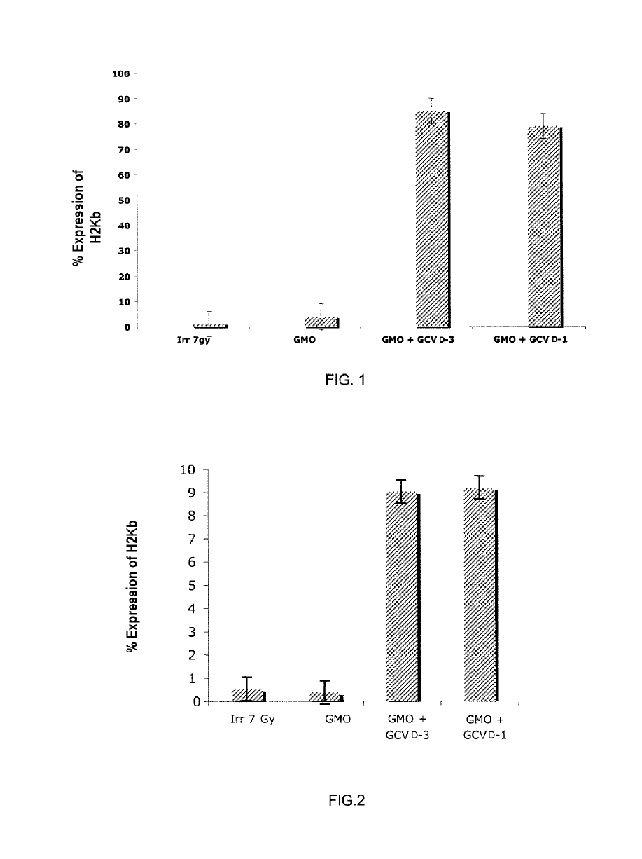

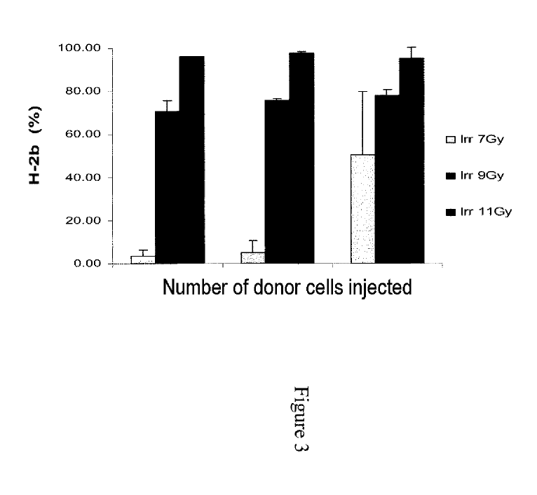

To study the chimerism of the recipient mice at D+27 (FIG. 1) and D+57 (FIG. 2), D0 being the day of the transplantation of haematopoietic stem cells, the percentage expression of H2 Kb (cells of donor origin) and H2 Kd (cells of recipient origin) was measured. A correlation thereby exists between the percentage of H2 Kb and H2 Kd expressed and the establishment of the graft. The higher the percentage expression of H2 Kb, the greater the establishment of the graft. Conversely, the lower the percentage expression of H2 Kd, the lesser the establishment of the graft.

In the case of the recipient mice whose conditioning before the transplantation was limited to the irradiation of 7 Gy, a percentage expression of the H2 Kb gene of the order of 0% at D+27 and of the order of 5% at D+57 was observed. This percentage corresponds to a rejection of the graft.

For the group of recipient mice (B6xD2)F1H2^{-bxd} having received an injection of the composition of T lymphocytes C57B1/6TK and an injection of gancyclovir at D−3, the percentage of H2 Kb was more than 80% at D+27 and practically 100% at D+57. For the group of recipient mice (B6xD2)F1H2^{-bxd} having received an injection of the composition of T lymphocytes C57B1/6TK and an injection of gancyclovir at D−1, the percentage of H2 Kb was 80% at D+27 and practically 100% at D+57. This high percentage expression of H2 Kb corresponds to an excellent establishment of the graft. No sign of GVH was detected.

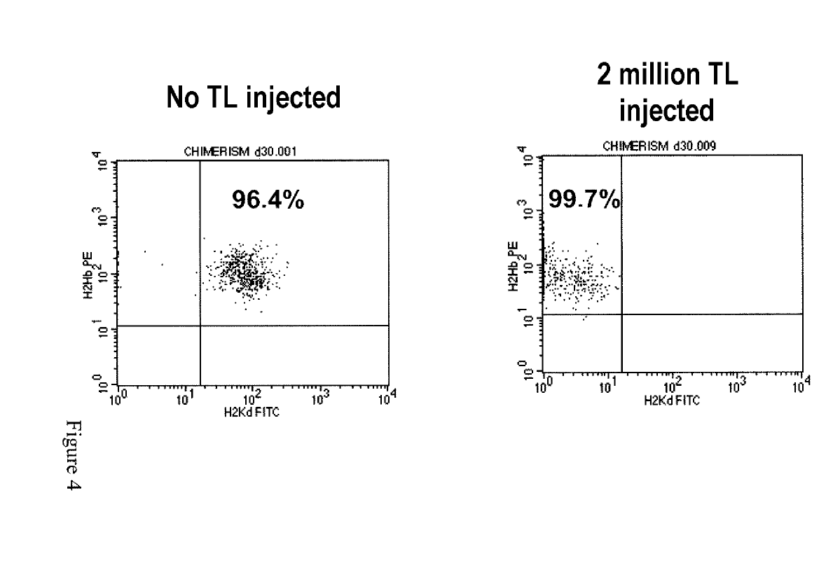

In another series of experiments, the recipient mice (B6xD2)F1H2^{-bxd} were exposed to a variable irradiation ranging from 7 to 11 Gy. A quantity of haematopoietic stem cells derived from the spinal cord of between 5×10⁵ and 2×10⁶ cells was collected from all the donor mice C57B1/6CD3e−/−. The results are presented in FIG. 3 and illustrate the excellent establishment of the graft in the animals treated. In particular, it is observed that at 9 and 11 Gy, there is a practically complete establishment of the graft regardless of the number of medullary cells injected.

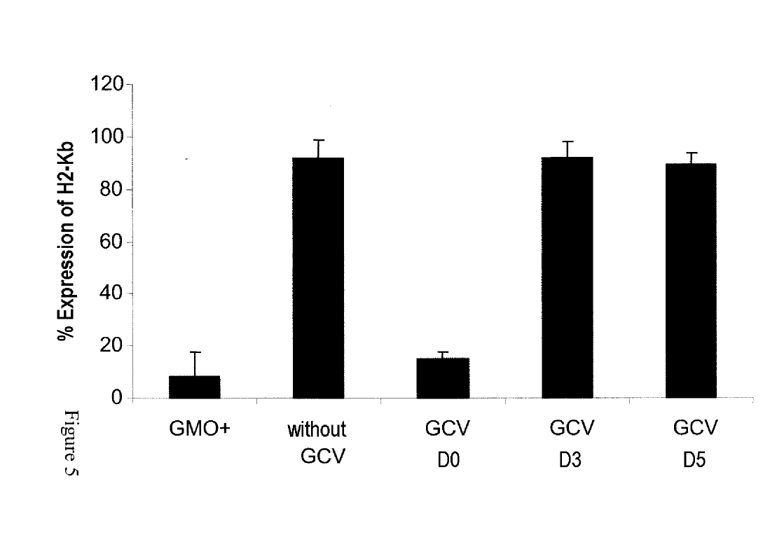

In an additional experiment, 2×10⁶ cells obtained from the peripheral ganglia and containing about 80% of T lymphocytes were added to the 2×10⁶ bone marrow cells injected. As shown in FIG. 4, the addition of the allogenic T lymphocytes makes it possible to arrive at 99.7% of cells of donor origin, but 100% of the mice develop a fatal GVH. On the other hand, when T lymphocytes obtained from TK+ transgenic mice are added on the day of the transplantation of bone marrow, and the mice are subjected to a treatment with ganciclovir (GCV) at D0, D3 or D5 for 2 days, it is possible to considerably improve the establishment of the allogenic graft. Accordingly, as shown in FIG. 5, without GCV, the establishment of the graft is practically complete but the mice die of GVH. The presence of GCV at D0 allows an effective control of the GVH, and the delayed administration of GCV (at D3 or at D5) allows a practically complete establishment of the graft without the mice developing clinical GVH.

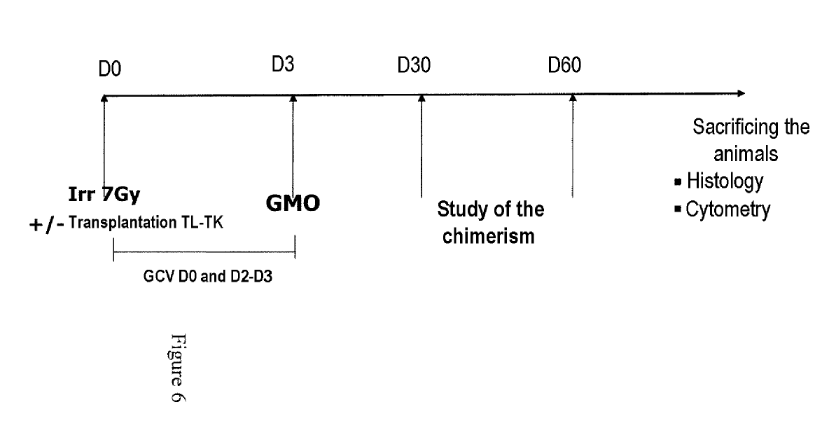

Another transplantation protocol is described in FIG. 6. According to this protocol, the moment of administration of the medullary cells is delayed. Accordingly, the treatment starts with a step of “conditioning the recipient” by the presence of alloreactive T lymphocytes for 3 days. Next, these T lymphocytes are removed by a treatment with ganciclovir. Transplantation of bone marrow is then performed.

Finally, we repeated the same type of experiment but this time the marrow cells, (BALB/c), the TK+ T lymphocytes (C57BL/6) and the recipient (C3H) are obtained from 3 different genetic pools. Under these conditions, when GCV is administered at D2, it was possible to obtain a partial establishment of the graft (mixed chimerism).

### BIBLIOGRAPHIC REFERENCES

Aversa et al, Treatment of High risk acute leukaemia with T-cell-depleted stem cells from related donors with one fully mismatched HLA haplotype. N Eng J Med, 1998; 339: 1186-1193.
Ciceri F, Bonini C, Marktel S, Zappone E, Servida P, Bernardi M, Pescarollo A, Bondanza A, Peccatori J, Rossini S, Magnani Z, Salomoni M, Benati C, Ponzoni M, Callegaro L, Corradini P, Bregni M, Traversari C, Bordignon C. 2007 Blood. 109(11):4698-707. Antitumor effects of HSV-TK-engineered donor lymphocytes after allogenic stem-cell transplantation.
Cohen et al, Suicide gene therapy of graft-versus-host disease: immune reconstitution with transplanted mature T cells. Blood, 2001; 98: 2071-2076.
Danos, O. and Mulligan R. C. 1988. Safe and efficient generation of recombinant retroviruses with amphotropic and ecotropic host ranges. Proc. Natl. Acad. Sci. USA 85, 6460-6464.
Kasid, A., Morecki, S., Aebersold, P., Cornetta, K., Culver, K., Freeman, S., Director, E. Lotze, M. T., Blaese, R. M. Anderson., W. F., and Rosenberg, S. A. 1990. Human gene transfer: characterization of human tumor-infiltrating lymphocytes as vehicles for retroviral-mediated gene transfer in man. Proc. Natl. Sci. USA 87, 473-477.
Markowitz, D., Goff, S., and Bank, A. 1988. Construction and use of a safe and efficient amphotropic packaging cell line. Virology 167:400-406.
Miller J S, Weisdorf D J, Burns L J, Slungaard A, Wagner J E, Verneris M R, Cooley S, Wangen R, Fautsch S K, Nicklow R, Defor T, Blazar B R. 2007 Blood.110(7):2761-3, Lymphodepletion followed by donor lymphocyte infusion (DLI) causes significantly more acute graft-versus-host disease than DLI alone.
Petersen et al, Alloreactivity of therapeutic principle in the treatment of hematologic malignancies. Danish Medical Bulletin, May 2007, 54:112-39
Ratanatharathorn V et al, Phase III study comparing methotrexate and tacrolimus (prograf, FK506) with methotrexate and cyclosporine for graft-versus-host disease prophylaxis after HLA-identical sibling bone marrow transplantation. Blood, 1998; 92: 2303-2314.
Socie G et al, Acute graft-versus-host-disease. The Clinical Practice of stem-cell transplantation. Isis Medical Media: Oxford, 1998, pp 595-618.
Storb R et al, Methotrexate and cyclosporine versus cyclosporine alone for prophylaxis of graft-versus-host disease in patients given HLA-identical marrow for leukemia: long-term follow-up of a controlled trial. Blood, 1989; 73: 1729-1734.

1-12. (canceled)

13. A method for conditioning a recipient patient for a transplantation of haematopoietic stem cells, which method comprises injecting to said patient allogenic T lymphocytes expressing a molecule allowing their specific destruction, before transplantation of haematopoietic stem cells.

14. The method of claim 13, wherein the T lymphocytes express a transgene allowing their specific destruction.

15. The method of claim 14, wherein the transgene is a “suicide” gene.

16. The method of claim 15, wherein the “suicide” gene encodes a molecule capable of reacting with a nucleoside analogue in order to lead to the death of the said T lymphocytes.

17. The method of claim 16, wherein said molecule encoded by the “suicide” gene is a molecule capable of phosphorylating a nucleoside analogue to a monophosphate molecule, itself convertible by cellular enzymes to a triphosphate nucleotide that can be incorporated into nucleic acids during extension under the effect of polymerases, the effect being the interruption of chain extension.

18. The method of claim 17, wherein said molecule encoded by the “suicide” gene is thymidine kinase of the herpes simplex virus type 1.

19. The method of claim 13, wherein said T lymphocytes are obtained neither from the donor nor from the recipient.

20. The method of claim 13, wherein said haematopoietic stem cells are haematopoietic stem cells derived from the bone marrow, peripheral blood after mobilization or umbilical cord blood.

21. The method of claim 13, wherein the injection of the T lymphocytes is performed 1 to 15 days before the transplantation of haematopoietic stem cells.

22. The method of claim 13, wherein the injection of the T lymphocytes is followed by destruction of said T lymphocytes before the transplantation of haematopoietic stem cells.

23. The method of claim 22, wherein the T lymphocytes express the thymidine kinase gene, and the injection of the T lymphocytes is followed by administration of gancyclovir or acyclovir before the transplantation.

24. A method for transplanting haematopoietic stem cells of a donor into a recipient patient, comprising a) injecting the patient with allogenic T lymphocytes expressing a molecule allowing their specific destruction, and b) transplanting haematopoietic stem cells into the patient.

25. The method of claim 24, wherein the T lymphocytes express a transgene allowing their specific destruction.

26. The method of claim 25, wherein the transgene is a “suicide” gene.

27. The method of claim 26, wherein the “suicide” gene encodes a molecule capable of reacting with a nucleoside analogue in order to lead to the death of the said T lymphocytes.

28. The method of claim 27, wherein said molecule encoded by the “suicide” gene is a molecule capable of phosphorylating a nucleoside analogue to a monophosphate molecule, itself convertible by cellular enzymes to a triphosphate nucleotide that can be incorporated into nucleic acids during extension under the effect of polymerases, the effect being the interruption of chain extension.

29. The method of claim 28, wherein said molecule encoded by the “suicide” gene is thymidine kinase of the herpes simplex virus type 1.

30. The method of claim 24, wherein said T lymphocytes are obtained neither from the donor nor from the recipient.

31. The method of claim 24, wherein said haematopoietic stem cells are haematopoietic stem cells derived from the bone marrow, peripheral blood after mobilization or umbilical cord blood.

32. The method of claim 24, wherein the injection of the T lymphocytes is performed 1 to 15 days before the transplantation of haematopoietic stem cells.

33. The method of claim 24, wherein the injection of the T lymphocytes is followed by destruction of said T lymphocytes before the transplantation of haematopoietic stem cells.

34. The method of claim 24, wherein the T lymphocytes express the thymidine kinase gene, and the injection of the T lymphocytes is followed by administration of gancyclovir or acyclovir before the transplantation.
